(19) United States
(12) Patent Application Publication
(10) Pub. No.: US 2017/0367678 A1
(43) Pub. Date: Dec. 28, 2017

(54) ULTRASOUND AUTOMATED METHOD FOR MEASURING THE THICKNESS OF THE WALLS OF THE LEFT ANTERIOR DESCENDING, RIGHT AND CIRCUMFLEX CORONARY ARTERIES

(71) Applicants: Cesare SIRTORI, Milano (IT); Dionysis GOULARAS, Istanbul (TR); Fabien LABOMBARDA, Herouville Saint Clair (FR)

(72) Inventors: Cesare SIRTORI, Milano (IT); Dionysis GOULARAS, Istanbul (TR); Fabien LABOMBARDA, Herouville Saint Clair (FR)

(21) Appl. No.: 15/189,864

(22) Filed: Jun. 22, 2016

(51) Int. Cl. A61B 8/08; A61B 8/00

(52) U.S. Cl. CPC A61B 8/0891; A61B 8/5246; A61B 8/5207; A61B 8/085; A61B 8/461; A61B 8/4483; A61B 8/488

(57) ABSTRACT

Provided is an ultrasound automated method for measuring the thickness of the walls of arteries. The method includes utilizing an apparatus that includes an ultrasonic device, a tracking system, a QI system, a 3D generator, a cross-section generator, a display driver, and a display. The ultrasound automated method includes, e.g.,: defining a fixed frame of reference, detecting the position of the ultrasonic device in respect to the fixed frame of reference, recording a set of 2D images, to be transmitted by the ultrasonic device to the database of the QI system structure, placing the reference sensor over the arterial walls, selecting the proximal and distal walls of the arteries, selecting the length of the walls to be measured in order to proceed to thickness determination, investigating over the length of the selected coronary segment, the mean arterial thickness (AT) and lumen area (LA).

### FIELD OF THE INVENTION

The present invention relates to a method for ultrasound image acquisition, allowing direct measurement of the coronary wall thickness of the two major coronaries, i.e. the left anterior descending coronary artery (LAD) and right coronary artery (RCA) and, when accessible, the Circumflex (Cx) coronary artery. The present invention relates to an ultrasound automated method for measuring the thickness of the walls of said arteries.

Said ultrasound automated method is commonly implemented by means of an apparatus comprising a tracking system, including an ultrasonic device, a QI (quality image) system, a 3D generator, a cross-section generator, a display driver and a display.

DESCRIPTION OF THE PRIOR ART

Coronary arterial disease is the major cause of death in the Western world. Occurrence of events, such as myocardial infarction (MI), angina or other coronary related diseases can be predicted based on a number of clinical evaluations, such as clinical and laboratory indicators of risk (blood pressure, cholesterol, smoking and others), cardiac stress tests and a variety of imaging evaluations. These evaluations can be developed from PET scans of cardiac arterial flow, CAT or MRI imaging of the coronary circulation, to invasive procedures, such as coronary angiograms, eventually leading to coronary procedures, PTCA or coronary bypass. These procedures are frequently accompanied by exposure of the patients to radiations, have a high cost and mostly do not allow a direct evaluation of the coronary artery wall. This is particularly relevant since the observation of raised arterial wall thickness may be associated to larger coronary plaques, possibly presenting with instability and eventual rupture, leading to coronary events.

The ultrasound automated method for measuring the thickness of the walls of the left anterior descending, right and circumflex coronary arteries aims to provide an up to date, high sensitivity method to investigate wall characteristics of the major coronary arteries.

These arteries can be, in fact, directly visualized by transthoracic echocardiography (TTE). This type of evaluation has been, however, hampered by the poor quality of available probes up to some years ago and by the lack of an appropriate software allowing to investigate cross-sections of the arterial lumen and thickness of the wall.

Wall thickness appears to be a very significant index, predicting overall coronary disease risk. It has been clearly noted that the presence of wall damage in a coronary (thickening, plaque, with or without superficial erosion) is associated with at least an 80% risk of having a number of other coronary alterations (McPherson et al. N Engl J Med 1987; 316: 304-9). The capacity to directly measure wall thickness appears to provide a direct evaluation of coronary artery conditions. Preliminary data indicate that an increase thickness, particularly of LAD, can be associated to an increased cardiovascular risk (Perry R, et al Echocardiography 2013; 30: 759-64).

The addition to this sensor system of a dedicated software, evaluating cross-sections of the wall thickness for a length of approximately 3-4 cm, further enhances the capacity of evaluating coronary risk and, possibly the effect of different therapies on this important coronary parameter.

In particular, drug treatment adopted for lipids reduction, or HDL-C increase may have impact on coronary wall thickness, in a similar way as shown for carotid intima media thickness (CMT), a vastly used diagnostic methodology (Baldassarre D, et al. Arterioscler Thromb Vasc Biol 2013; 33:2273-9), that however has not always provided reliable results in terms of cardiovascular risk prediction (Naqvi T Z, Lee M S. JACC Cardiovasc Imaging 2014; 7:1025-38).

### SUMMARY OF THE INVENTION

The present invention is intended to be used in the medical diagnostic framework as the body to be imaged and recorded is comprehensive of anatomical structures.

The present invention bases its evaluation on the position and orientation of a probe in the coronary system, comprising a fixed field transmitted thus defining a framework of reference and an anatomical structure sensed by the probe.

The aim of the invention is to overcome the problems of presently available methods, that allow only a direct measurement of coronary wall thickness but are exposed to the manual experience of the operator, who needs to be able to keep the probe device in a fixed position in the presence of heart movements.

According to the invention the ultrasound automated method is implemented by means of an apparatus.

The apparatus comprises a tracking system, including an ultrasonic device, a QI system, a 3D generator, a cross-section generator, a display driver and a display, Furthermore the method comprises a first step, defining a fixed frame of reference, a second step wherein the tracking system detects the position of the ultrasonic device in respect of the fixed frame of reference, a third step, wherein the tracing system records a set of 2D images, to be transmitted to the database of said QI system structure, a fourth step, wherein the reference sensor is placed over the arterial walls of the left anterior LAD and RCA, a fifth step, wherein the QI system selects the length of the two walls to be measured and a sixth step, wherein a software allows to investigate over the length of the selected coronary segment, the mean arterial thickness (AT) and lumen area (LA).

### BRIEF DESCRIPTION OF THE DRAWINGS

The above and other objects, features and advantages of the present invention will become more apparent from the following description of preferred embodiments in connection with the accompanying drawings, wherein:

the FIG. 1 shows a scheme of the method of the present invention, it shows in particular all the six steps comprised within said method;

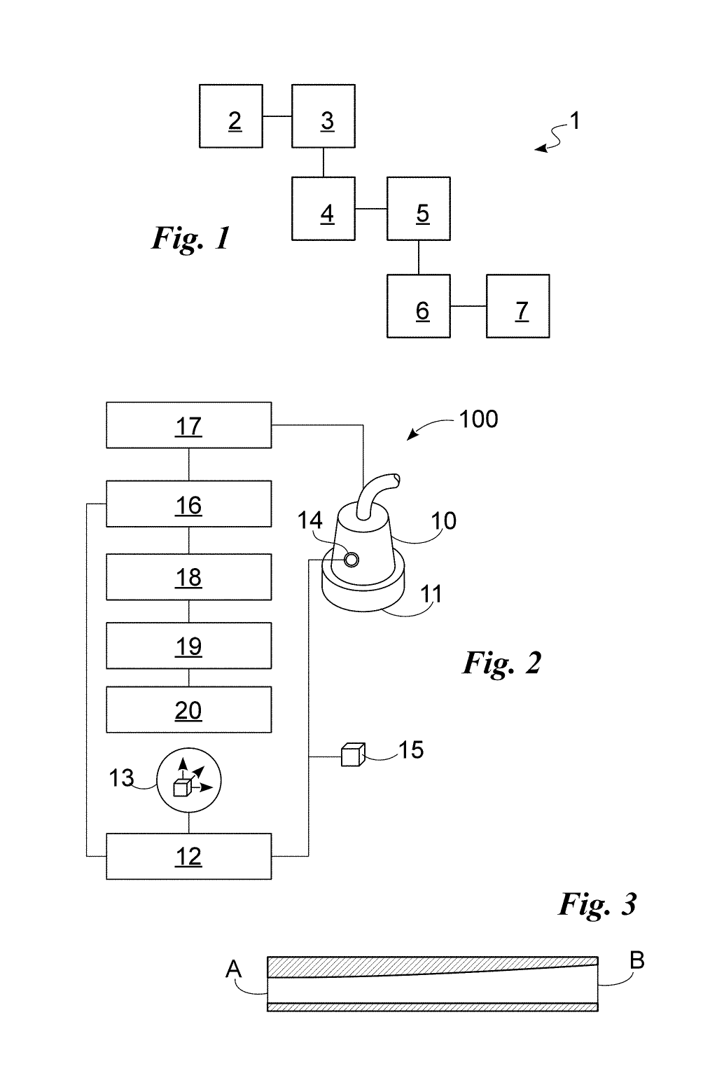

the FIG. 2 shows a scheme of the apparatus of the present invention; it shows in particular an ultrasonic device for ultrasound image acquisition, comprising an ultrasound probe having at least one piezoelectric transducer for transmitting the ultrasonic signal and for receiving and processing the signal of echography, as well as one transducer;

the FIG. 3 shows the creation of the cross section for segments A and B corresponding to the top and bottom section of the artery;

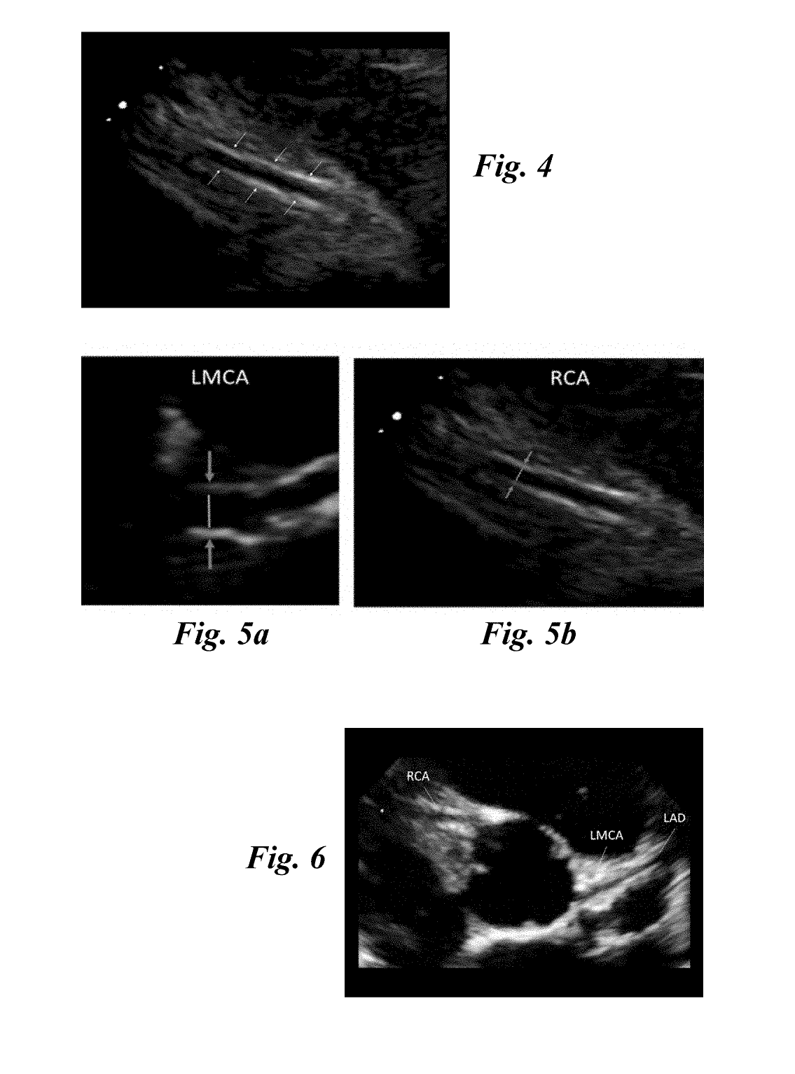

the FIG. 4 shows the appearance of a normal right coronary artery by ultrasound;

the FIG. 5a shows the determination of coronary wall thickness in the left main coronary artery (LMCA);

the FIG. 5b shows the determination of coronary wall thickness in the right coronary artery (RCA);

the FIG. 6 shows the detection of left main coronary artery (LMCA) and left arterial descending coronary artery (LAD) after emergence from the ascending aorta and right coronary artery (RCA);

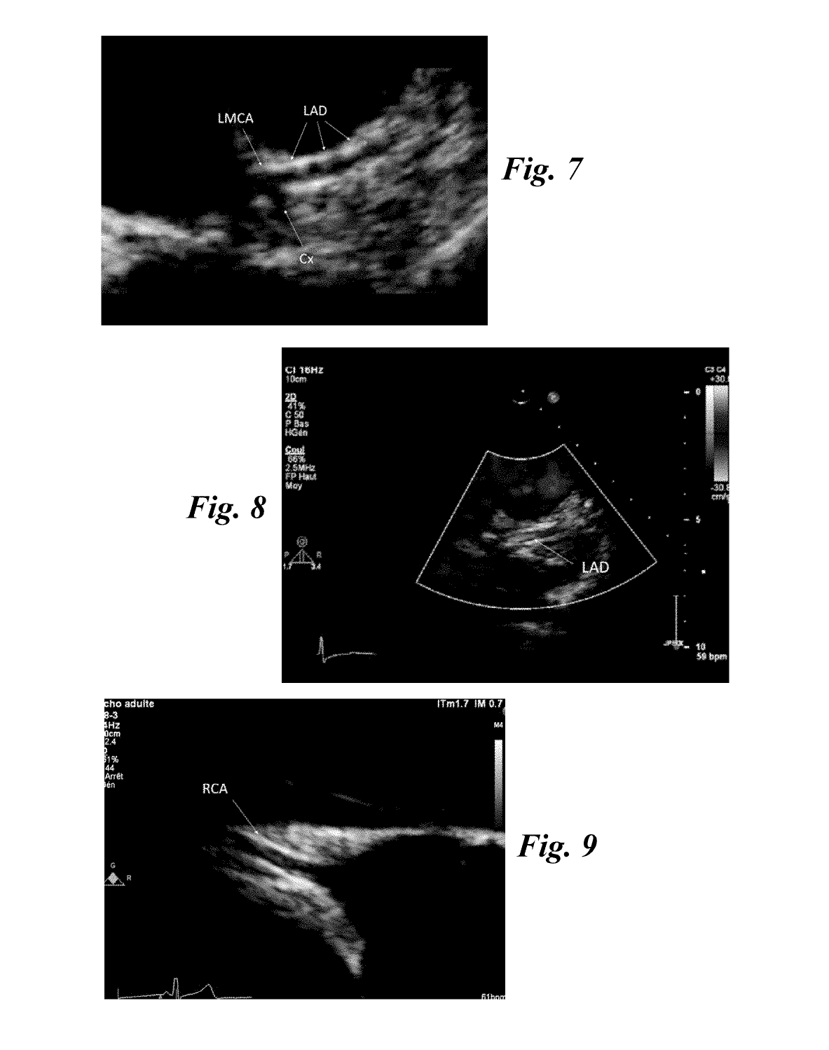

the FIG. 7 shows the bifurcation of the LMCA to LAD and circumflex coronary artery (Cx);

the FIG. 8 shows the ultrasound evaluation with doppler in order to assess blood flow in the LAD;

the FIG. 9 shows the ultrasound evaluation of segment of the right coronary artery (RCA);

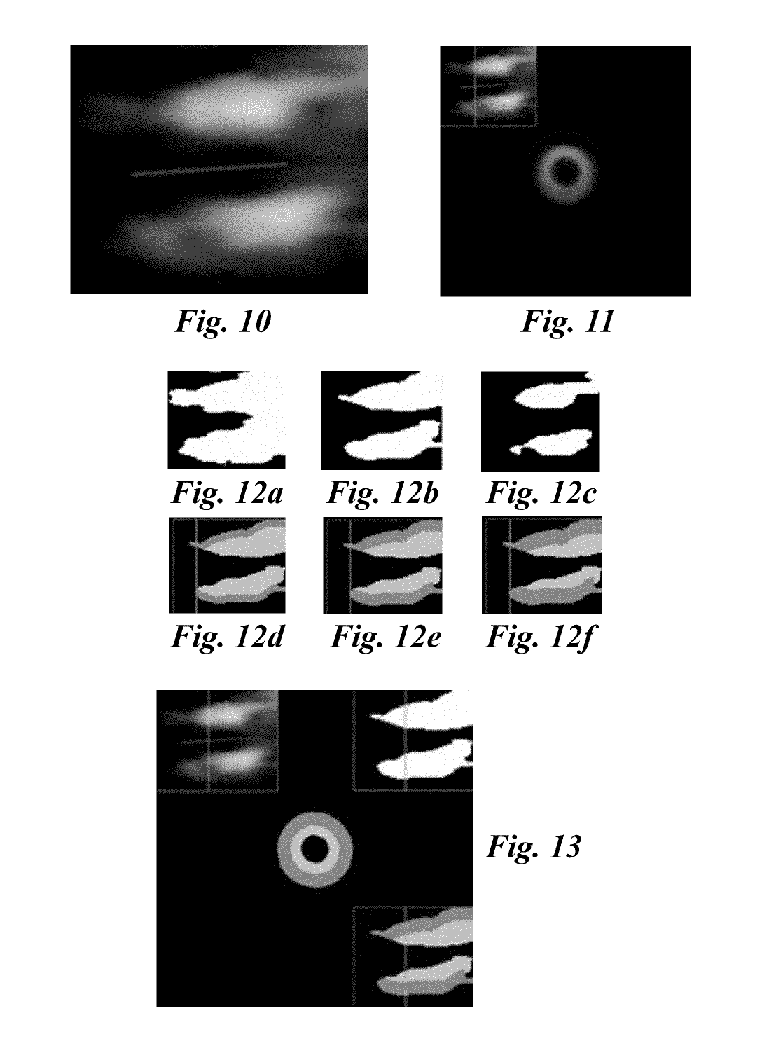

the FIG. 10 shows the center axis of coronary artery as defined and drawn by the user (red line);

the FIG. 11 shows the generation of a cross section from a vertical segment of a longitudinal section (green line);

the FIGS. 12a to f shows representations of the arterial wall and plaque, the method depending on selected progressive threshold values and arterial wall thicknesses;

the FIG. 13 shows the original longitudinal section (top left), section after applying a threshold value (top right), section after applying a wall thickness (bottom right), cross section (center).

### DESCRIPTION OF THE PREFERRED EMBODIMENTS

The present invention relates to an ultrasound automated method 1 for measuring the thickness of the walls of the left anterior descending and right coronary arteries, i.e. left anterior descending coronary artery (LAD) and right coronary artery (RCA).

Referring to FIG. 2 ultrasound automated method 1 is implemented by means of an apparatus 100.

Said apparatus 100 comprises a tracking system 12, a QI system 16, a 3D generator 17, a cross-section generator 18, a display driver 19, a display 20.

An example of the tracking system 12 is produced by ESAOTE with the trademark of MyLab Eight.

An example of the QI system 16 is produced by Siemens with the trademark of ACUSONX300.

An example of the 3D generator 17 is produced by Philips with the trademark of iE33-3D.

The tracking system 12 comprises an ultrasonic device 10, a transmitter 13 and a reference sensor 15.

The ultrasonic device 10 comprising at least one probe sensor 14.

The probe sensor 14 is preferably a piezoelectric transducer adopted to transmit the ultrasonic signal and to receive and process the signal of echography.

Ultrasonic device 10 is 2D type and acquires subsequent 2D images in order to eventually generate 3D images from a scan volume 11. An example of the scan volume 11 is produced by Philips with the trademark of EPIQ Ultrasound.

The scan volume 11 defines an acquisition surface which can be typical of common echography probes.

The tracking system 12 allows the probe sensor 14 coupled to the ultrasonic device 10 in a fixed way to communicate with the transmitter 13 and the reference sensor 15.

The transmitter 13 defines the fixed frame of reference thus probe sensor 14 detects position and orientation with respect to the said fixed frame of reference.

According to a preferred embodiment, the transmitter 13, probe sensor 14 and reference sensor 15 are of electromagnetic type. According to a further embodiment the acquisition is performed by means of a 2D device 10.

The reference sensor 15 is coupled to a QI system 16.

The QI system 16 indicates the system whereby the AT is calculated, provides the error of the determination and memorizes data in a database.

The QI system 16 allows to generate a series of 2D images, providing the final one to be evaluated by the cross-section generator 18, or generating the 3D image in the 3D generator 17.

The cross-section generator 18 realizes a cross-section from the last image of the sequence of images combined in previous QI system 16.

The 3D generator 17 realizes a 3D imagine using the effect of panoramic combination adopted by QI system 16.

The display driver 19 may be a common video device, i.e. a monitor, adopted to show on a display 20 the video images captured with the ultrasound automatic method 1 so that these video images can be examined by the operator, thus providing a diagnostic conclusion on arterial wall thickness and coronary lumen area.

Thus the display 20 may be a common screen of a monitor.

The ultrasound automated method 1 is particularly advantageous for the detection of vascular abnormalities in the coronaries. In an embodiment the acquisition is generally performed by means of a ultrasonic device 10 2D type. Preferably the acquisition by means of the ultrasonic device 10 2D type is performed manually. In particular, the trans thoracic echocardiographic (TTE) technique of examination for the proximal segments of main coronary arteries can be standardized. Several echocardiographic windows can be used for visualization of the coronary arteries with the patient in the supine or left decubitus positions. Standard parasternal short- and long-axis views from second- or third intercostal space or low parasternal short- or long-axis views from fourth- or fifth intercostal space should be used. A modified apical two, three or five-chamber view can alternatively be performed. The scanning depth for the search of proximal coronary artery segments should begin at 10-15 cm. Coronary arteries appear as linear intra-myocardial structures of approximately 1-5 cm in length and 2 to 4 mm in diameter.

The probe sensor 14 should be placed at the left parasternal position from second or third intercostal space and a modified short-axis view of great vessels should be obtained. Initially, a short part of arteries can be visualized. Then, by step by step movement of the transducer, i.e. the probe sensor 14, according to the course of the vessel, a longer segment can be assessed. The search of the left main coronary artery (LMCA) and proximal LAD can be started in two-dimensional-mode (2D) by consecutive clockwise and cranial rotation of the transducer; color Doppler mapping can also be recommended for initial search. The LMCA is of approximately 2-5 cm in length, and the vessel should be visualized along its entire extension.

The circumflex artery surrounds, instead, the anatomical location of the mitral valve, allowing to see the middle third of the artery.

The normal anterograde blood flow in the LMCA and LAD is identified on color Doppler map as a linear structure dawning from the left coronary sinus of Valsalva. Bifurcation of the vessel into the LAD and circumflex coronary artery (Cx) is a marker of LMCA distance.

Proximal LAD should be assessed after the LMCA by a slight change of the imaging plane in a parasternal or low parasternal short-axis B-view or by change of the position in a modified parasternal long-axis view. The origin of the first diagonal branch can be used as a distal mark of proximal LAD.

The proximal right coronary artery (RCA) should be examined in the left parasternal position from second- or third intercostal spaces in modified short- or long-axis 2D-views as a structure dawning from right coronary sinus of Valsalva and lying along the anterior wall of the aorta. The first segment of the RCA is of approximately 1-3 cm in length, and should be visualized in its entirety.

The standard manual acquisition with a scanning device 2D type would allow a slower acquisition, in order to appropriately reconstruct the vessels without artifacts in Doppler mode. In fact during Doppler scans a scanning device cannot move fast enough, due to limited ultrasound Doppler frame rate, and the possibility of artifacts due to device movements.

However high quality is important since vessels to be imaged are thin.

According to the invention, images are fused by means of an automatic registration algorithm, matching vessels comprised in the panoramic 3D image, identified by segmentation of the volumetric image acquired in the different imaging modality.

This acquisition is performed manually by the operator, who detects within the panoramic 2D image an anatomical marker such as the ascending aorta or the heart septum. By this methodology, further acquired ultrasound 2D images can be combined with the first 2D images to form 3D images. They are thus automatically registered and can be treated a single image, allowing to calculate the arterial wall volume and arterial lumen by the above described software, as well by using algorithm, hereafter describe, allowing to generate multiple cross-sections.

The ultrasound automated method 1 comprises the following steps:
First step 2 wherein the transmitter 13 defines a frame of reference, including the two major arteries, as visualized by high frequency ultrasound transducers 10;
Second step 3 wherein the tracking system 12 detects the position and orientation of the frame to be imaged, thus the position of the probe sensor 14, with respect to the frame of reference, using an appropriate sensor system;
Third step 4 wherein the tracing system 12 records a set of 2D images, to be transmitted by an ultrasonic device 10 to the apparatus 100 structure and receiving the signal of echography;
Fourth step 5 wherein a reference sensor 15 is placed over the arterial walls of the left anterior LAD and RCA, selecting the proximal and distal walls of the two arteries;
Fifth step 6 wherein an appropriate QI system 16 selects the length of the two walls to be measured in order to proceed to thickness determination;
Sixth step 7 wherein an appropriate software allows to investigate over the length of the selected coronary segment, the mean arterial thickness (AT) and lumen area (LA).

In details, the first step 2 provides for example a fixed frame of reference adapted to allow the 2D device 10 to define its position in respect of the two major arteries. This first step 2 may be achieved with a common transmitter 13 defining a fixed frame of reference including the left anterior descending (LAD) and right coronary artery (RCA), as visualized by high frequency ultrasound transducers, i.e. probe sensor 14. The definition of the frame of reference begins from a standardized echocardiographic examination with careful interrogation of the aortic sinuses. The LAD arises at approximately at 4 o'clock and the RCA at 12 o'clock if you consider the aortic root as a clock face. The Cx is visible as surrounding the anatomical location of the mitral valve. The coronary arteries appear as linear intra-myocardial color fragmental structures of approximately 0.5-3.5 cm in length and 2 to 4 mm in diameter.

The second step 3 is made preferably for detecting position and orientation of the frame to be imaged by reference sensor 15 with respect to the frame of reference by transmitter 13, using an appropriate sensor system. The criterion used to define the position and orientation of the frame is based on the optimal detection of the two hyper-echogenic linear echoes of the coronary arterial walls.

The offered system has an appropriate memory allowing a rapid recognition of the required frame.

The second step 3 is implemented by means of the ultrasonic device 10 coupled with probe sensor 14 with at least one piezoelectric transducer, and a stage for transmitting an ultrasonic beam by at least one transducer into a body to be imaged. It is also comprehensive of a stage for receiving and processing signals of echography returned from at least one transducer.

The position and orientation of the reference probe 15 defines the frame to be imaged.

The operator working with the device 10 is responsible for it so that the correct orientation may be subject to “human factor” problems. However, most problems can be solved by providing the tracking system 12 with the said transmitter 13 defining the fixed frame of reference, that can detect the position of the probe sensor 14 coupled to the ultrasonic probe 10. This sensor can detect position and orientation of the device 10 with respect to the fixed frame of reference.

Thanks to the ultrasonic device 10 it is possible to proceed with the subsequent steps.

The third step 4 preferably consists in recording a set of 2D images of the LAD, RCA and Cx.

2D ultrasound images are obtained by a 2D ultrasound device 10. A large number of 2D ultrasound images are captured successively by shifting the device 10 and transmitted by a probe sensor 14 to the apparatus 100 structure and receiving the echographic signal for image processing operations.

The fourth step 5 is implemented by means of a reference sensor 15 to be positioned over the arterial walls of LAD and RCA, selecting the proximal and distal walls of the two arteries. Measurement of arterial wall thickness will be obtained by an appropriate software, providing information also on the eventual progression/regression of disease.

The fifth step 6 is implemented by means of a QI system 16 appropriate for the selection of the length of the two walls to be measured, in order to proceed to thickness determination as indicated in the previous step of said method 1.

Eventually the sixth step 7 is implemented by means of an appropriate software allowing to investigate over the length of the selected coronary segment, the mean arterial thickness (AT) and lumen area (LA).

The software is based on the analysis of a single image extracted from the ultrasound device 10 representing a longitudinal section of the LAD or RCA. After isolating the artery from the rest of the initial image, the analysis starts with a threshold-based segmentation procedure aiming to keep only the regions of interest.

Then, a wall thickness is defined, based on the metrics of the image and the standard wall thickness. Finally, using adequate algorithms, cross-sections of the coronary artery are generated based on the top and bottom wall width that are extracted from the longitudinal section, allowing thus the calculation of the plaque thickness in different parts of the artery.

The fifth step 6 comprises, as already said, an algorithm for the generation of multiple lateral cross-sections from a single longitudinal coronary artery section. The generation of cross-sections from a single longitudinal coronary artery section can be achieved in fifth step 6 with two different procedures, resulting a simple gray level representation and a more analytical representation including the wall arteries and plaque respectively.

In a first example the generation of cross-sections from a single longitudinal coronary artery section is achieved by means of a Gray level representation of artery's cross sections.

In such an example, the algorithm that generates multiple lateral cross-sections from a single longitudinal coronary artery section comprises the following proceedings: at first, a user should define manually on the image of the longitudinal section the center axis of the artery. This operation is relatively simple: the user draws with the mouse a simple curve (spline) that can be further adjusted, as shown in FIG. 10. Then, the image is scanned from left to right and for each vertical column with one pixel width, the upper and lower wall regions of the artery are detected. Each region is defined by the distance between the center axis and the top of the image for the upper wall and the bottom of the image for the lower wall, respectively. Then, a set of intermediate values is generated between the segment of the upper wall and the segment of the lower wall by applying a linear interpolation between the values of the two segments in order to ensure that a smooth transition is carried out. Then, the generated values are circularly projected resulting in a cross section, as shown in FIG. 11. The advantage of this method is that a physician can have very quickly a first qualitative diagnosis of the general condition of the artery.

In a second example the generation of cross-sections from a single longitudinal coronary artery section is achieved by means of a representation of the wall artery and plaque:

This method requires again that the user defines the center axis of the artery as described in the previous method. Then the user should perform two additional proceedings: selection of a threshold value for the artery representation and selection of the width of the wall artery.

The Selection of a threshold value for the artery representation defines that the user will have an option to select a value in order to decide which part of the image will be chosen for the generation of the artery. Different threshold values result in different representations of the artery as shown in FIGS. 12a, 12b, 12c, 12d, 12e and 12f. This step is important and the experience of the user may be critical in order to select a representation corresponding best to the artery. The operation is performed in a very short period of time (less than a minute) and quite easy, by using a simple method like a slider and/or a text box where the threshold value can be inserted

The selection of the width of the wall artery for example comprises a proceeding wherein the thickness of the arterial wall can be chosen automatically based on the artery diameter according to standard measurements. Nevertheless, the user will be able to modify the thickness with the help of a slider or text box, according to his experience.

When the user decides about the threshold and wall artery values, cross-sections are generated from corresponding segments from the top and bottom part of the artery. Initially, these sections will be ‘filled’ with a white color as shown in FIG. 3. The borders are not connected with a straight line but with a curve (spline).

The curviness is increasing as long the segments are of different size, resulting a shape with respect to the artery's natural shape. Then, this segment is projected circularly in order to create the cross section and finally, the artery wall and plaque are drawn as shown in FIG. 13. After the generation of the cross sections it is possible to calculate the internal diameter of the artery in different positions. Furthermore, as a 3D model can be extracted, additional calculations related to blood flow and speed may be also calculated.

The system has an appropriate recording system storing images and allowing repeated assessments with confrontation of earlier scans, thus providing an evaluation of the clinical progression or regression of coronary disease as comprised in the sixth step 7.

After the creation of the cross sections two presentations can be generated:
a video on the display of 20 presentation resulting from the generated cross-section images, utilized as continuous frames
In order to achieve the 3D presentation a 3D model is created by the 3D generator 17 from the generated cross-sections. Then, the real time rendering of stereo images generated from the 3D model will allow to visualize and navigate in stereo vision in real-time inside the artery. This will thus provide a tool for analysis and for diagnostic purposes. The manipulation of the 3D artery model in stereo vision will allow an improved understanding of the arterial status compared to the traditional methods of image and video visualization. The cardiologist will be able to manipulate the artery in the Virtual Environment, similar to the real world by performing actions like rotate, translate or zoom.

Additionally, the physician will have the possibility to remove according to an axis parts of the artery in order to better visualize the sections or cross sections of the artery at a specified area.

The virtual reality application will be compatible with existing technologies such as Oculus.

The ultrasound automated method for measuring the thickness of the walls of the left anterior descending and right coronary arteries shows important advantages. An operator can overcome the problems of presently available methods, that allow only a direct measurement of coronary wall thickness, thus available methods are exposed to the manual experience and error of the operator, who needs to be able to keep the probe device in a fixed position in the presence of heart movements. Differently method 1 is preferably characterized by a method comprising the device 10 which allows to solve said previous problems by providing a tracking system which comprises a transmitter 13 defining a fixed frame of reference, that can detect the position of the probe sensor 14 coupled to the device 10. In this way multiple acquisitions in Doppler mode of the 2D images constituting 3D images can be performed.

Another important advantage is defined by the fact that method 1 allows the acquisition of ultrasound 2D images to be combined to form a 3D image, automatically registered as AT, or treated as single images registered in order to be able to calculate a mean arterial wall volume.

The invention is susceptible to variations comprised within the scope of the inventive concept defined by the claims. In this context all details are replaceable by equivalent elements and the materials; the shapes and the dimensions may be any.

1.-9. (canceled)

10. An automated ultrasound method for measuring thickness of left anterior descending (LAD), right (RC) or circumflex coronary artery walls with an ultrasonic device comprising
an ultrasonic device,
a tracking system comprising said ultrasonic device,
a QI system,
a 3D generator,
a cross-section generator,
a display driver, and
a display,
said method comprising:
defining a fixed frame of reference;
detecting, with said tracking system, the position of said ultrasonic device in respect to said fixed frame of reference;
recording, with said tracking system, a set of 2D images to be transmitted by said ultrasonic device to the database of said QI system structure;
placing said reference sensor over proximal and distal walls of the arteries;
selecting, with said QI system, the length of the artery walls to be measured in order to proceed to a thickness determination;
investigating, over the length of the selected coronary segment, the mean arterial thickness (AT) and lumen area (LA).

11. The automated ultrasound method of claim 10, wherein said QI system transmits said 2D images to said cross-section generator and said 3D generator, said cross-section generator generating an image of coronary cross sections and said 3D generator generating a visual pattern by multiple images of the panoramic view of LAD and RC coronaries.

12. The automated ultrasound method of claim 10, wherein said tracking system comprises
a probe sensor positioned within said ultrasonic device,
a transmitter defining said fixed frame of reference,
a reference sensor defining a frame to be imagined.

13. The automated ultrasound method of claim 12, wherein said probe sensor is configured to
(a) scan standard parasternal short and long-axis from second and third intercostal space or from fourth or fifth intercostal space,
(b) move in a consecutive clockwise and cranial rotation while scanning,
(c) scan the circumference of an anatomic mitral valve.

14. The automated ultrasound method of claim 12, wherein said probe sensor comprises at least one piezoelectric transducer, transmitting an ultrasonic beam into a body to be imaged.

15. The automated ultrasound method of claim 10, wherein the first step defines said fixed frame of reference by means of said transmitter, said fixed frame including the two major arteries, as visualized by said ultrasonic device.

16. The automated ultrasound method of claim 10, wherein said tracking system detects the position of said probe sensor and said reference sensor in respect to said transmitter.

17. The automated ultrasound method of claim 10, wherein said selecting further comprises
defining manually a center axis of the artery on an image of a longitudinal section of said artery,
scanning the image of the longitudinal section of said artery from left to right and for each vertical column with one pixel width,
detecting upper and lower wall regions of the artery defined by the distance between the center axis and the top of the image for the upper wall and the bottom of the image for the lower wall, respectively,
applying a linear interpolation between the values of the two segments
projecting circularly the generated values resulting in a cross section.

18. The automated ultrasound method of claim 10, wherein said 3D generator realizes a 3D model by rendering of stereo images allow to visualize and navigate in stereo vision in real-time inside the artery.
